(19) United States
(12) Patent Application Publication
(10) Pub. No.: US 2022/0093219 A1
(43) Pub. Date: Mar. 24, 2022

(54) RECRUITING OF PATIENTS FOR MEDICAMENT STUDIES

(71) Applicant: Bayer Aktiengesellschaft, Leverkusen (DE)

(72) Inventor: Christian Johannes MÜLLER, Leverkusen (DE)

(73) Assignee: Bayer Aktiengesellschaft, Leverkusen (DE)

(21) Appl. No.: 17/423,056

(22) PCT Filed: Jan. 13, 2020
(86) PCT No.: PCT/EP2020/050635

§ 371 Date: Jul. 14, 2021

(30) Foreign Application Priority Data
Jan. 18, 2019 (EP) 19152557.5

(51) Int. Cl. G16H 10/20; G16H 70/40

(52) U.S. Cl. CPC G16H 10/20; G16H 70/40

(57) ABSTRACT

The present invention relates to the recruiting of patients for one or more medicament studies. The subjects of the present invention are a method, a system and a computer program product for recruiting patients for one or more medicament studies with the aid of an individual identification feature that is applied to the packaging of a medicament.

### CROSS REFERENCE TO RELATED APPLICATIONS

This application is a national stage application under 35 U.S.C. § 371 of International Application No. PCT/EP2020/050635, filed internationally on Jan. 13, 2020, which claims benefit of European Application No. 19152557.5, filed Jan. 18, 2019.

### FIELD OF THE DISCLOSURE

The present disclosure concerns the recruitment of patients for one or more drug trials. The present disclosure provides a process, a system, and a computer program product for recruiting patients for one or more drug trials with the aid of a unique identifier applied to the packaging of a medicament.

### BACKGROUND OF THE DISCLOSURE

The term drug trials is understood as meaning studies in which the effects of medicaments are investigated or demonstrated in an interventional or non-interventional manner and in which the risk-benefit relationship or safety or effectiveness of the medicaments is ascertained. Drug trials are a prerequisite for the registration of a new medicament. The medicament is examined in various phases to establish whether it meets requirements and expectations, in phase I in healthy volunteers and in phases II and III in patients.

Once the competent regulatory authority has granted marketing authorization, the pharmaceutical company may market the medicament as set out in the marketing authorization. However, many medicaments also undergo trials even after their registration. In these studies, known as phase IV trials, further information is obtained on the tolerability, therapeutic efficacy, product profiling, and on the examination of specific questions (e.g. long-term tolerability, rare undesirable effects, etc.). When a licensed medicament is investigated in a new indication, the phase by definition reverts to phase II or III; such studies are sometimes also referred to as phase V studies.

It is usual for a sponsor (usually the pharmaceutical company) to pay all participants who take part in the trial. Those paid include at least the patients taking part and a physician (an “investigator”) responsible for the supervision of the patients.

So-called contract research organizations (CROs) act as intermediaries between the sponsor on the one hand and between the investigator and the patients on the other. CROs often undertake the entire management of the trial, including all necessary duties: these include for example the development of the trial protocol, the recruitment of patients and investigators and/or study sites, the agreement of contracts with the participants, the supervision of the execution of the trial, the collection and evaluation of data, the channeling of payment from the sponsor to the participants, etc.

For the recruitment of patients, CROs access internal patient databases or prospective patients are approached by doctors. There are also technical systems to aid patient recruitment (see for example US2008177573, WO2004092912, WO2015127245, WO2018224156).

However, the identification of suitable patients is still very time-consuming and cost-intensive. Many studies fail because it is not possible to find the necessary number of suitable patients. Often it is not willingness to take part, but the flow of information towards suitable patients that is lacking.

Based on the described prior art, those skilled in the art are faced with the technical problem of providing a solution for the recruitment of patients for drug trials, in particular for phase IV trials, with which a large number of prospective and suitable patients will be approached, with which patients may be easily enrolled in a study, and with which patient information may be easily and quickly retrieved and made available to the sponsor and/or to the contract research organization.

This problem is solved by the subjects of the independent claims. Preferred embodiments can be found in the dependent claims and also in the present description and the figures.

### SUMMARY OF THE DISCLOSURE

The present disclosure firstly provides a process comprising the following steps:
inputting of data of a unique identifier affixed to an individual pack of a medicament into a computer by a prospective participant in a drug trial,
identifying one or more drug trials on the basis of the inputted data,
displaying the one or more drug trials to the prospective participant.

The present disclosure further provides a system comprising
an input unit,
an output unit,
a control unit, and
a database, wherein the control unit is configured to cause the input unit to receive data of a unique identifier affixed to an individual pack of a medicament from a prospective participant in a drug trial,

wherein the control unit is configured to identify one or more drug trials in the database on the basis of the data received, wherein the control unit is configured to cause the output unit to display information on the one or more identified drug trials to the prospective participant.

The present disclosure further provides a computer program product comprising a data carrier and program code that is stored on the data carrier and that causes a computer, in the memory of which the program code has been loaded, to execute the following steps:
receiving data of a unique identifier applied to an individual pack of a medicament,
identifying one or more drug trials on the basis of the received data,
displaying information on the one or more drug trials to a prospective participant in the one or more drug trials.

The present disclosure further provides for the use of a unique identifier affixed to an individual pack of a medicament for the recruitment of participants for a drug trial, characterized in that a prospective participant inputs the data of the unique identifier into a computer, on the basis of the data one or more drug trials are identified, and information on the one or more drug trials is displayed to the prospective participant.

The disclosure is elucidated in detail hereinbelow without making a distinction between the subjects of the disclosure (process, system, computer program product, use). Rather, the elucidations that follow are intended to apply by analogy to all subjects of the disclosure, irrespective of the context (process, system, computer program product, use) in which they occur.

Where steps in a sequence are mentioned in the present description or in the claims, this does not necessarily mean that the disclosure is limited to the sequence mentioned. Instead, it is possible that the steps can also be executed in a different sequence or else in parallel to one another, unless one step builds upon another step, which by definition means that the step building upon the other is executed subsequently (but this will be clear in the individual case). The sequences stated thus represent preferred embodiments of the disclosure.

The present disclosure allows participants for a drug trial to be easily and efficiently recruited.

The term “participant” is understood as meaning a person who uses medical services (patient) and is taking part voluntarily in a drug trial. The term “participant” covers both male and female participants, the same applies to the terms “prospective participant” and “patient”.

The term “prospective participant” is understood as meaning a patient who is checking whether he/she is able to take part in a drug trial or for whom this is being checked. If he/she is him/herself willing to take part in a drug trial and meets the criteria for taking part in the drug trial, he/she can register him/herself in the drug trial. By registering, he/she becomes a participant. Registering includes his/her informed consent.

The term “drug trial” is understood as meaning a study in which the effects of one or more medicaments are investigated or demonstrated in an interventional or non-interventional manner and/or in which the risk-benefit relationship and/or safety and/or effectiveness of the medicament(s) is ascertained.

The drug trial for which patients are to be recruited is preferably a non-interventional study. A “non-interventional study” is a study in which findings from the treatment of persons with medicaments are analyzed using epidemiological methods; the treatment in such studies, including the diagnosis and monitoring, does not follow a predefined protocol, but is exclusively in line with medical practice; where the trial concerns a medicament subject to registration/marketing authorization, this is additionally in accordance with the information for its use specified in the registration/marketing authorization. The drug trial for which patients are to be recruited is preferably a phase IV study.

The term “medicament” or “medicinal product”, which has the same meaning, is understood as meaning a substance or substance composition that is intended for the curing or prevention of pathologies in humans, is suitable for influencing physiological functions, or facilitates a medical diagnosis. The medicament that is the subject of the trial is preferably an already-licensed medicament that is already being marketed by a pharmaceutical company and can thus be obtained by a patient, e.g. in a pharmacy. It is preferably a prescription-only medicament.

The recruitment according to the disclosure of patients for one or more drug trials takes place by means of a unique identifier affixed to the individual pack of a medicament.

According to Art. 54a (1) of the EU Falsified Medicines Directive (FMD) amending Directive 2001/83/EC, at least prescription-only medicaments must be identified with a unique identifier that in particular facilitates checking that a product is genuine and the identification of individual packs.

Details regarding the characteristics and technical specifications of the unique identifier were laid down in Commission Delegated Regulation (EU) 2016/161 of 2 Oct. 2015 and published in the Official Journal of the European Union on 9 Feb. 2016.

According to Art. 4 of the Delegated Regulation, the unique identifier comprises the following data elements: product code, serial number, batch number, and expiration date. This information is also shown in plain text on the individual pack.

The medicament may be specified above the product code, i.e. it is possible to establish what medicament it is.

The combination of product code and serial number is unique worldwide for every pack of the medicament (for a defined period at least).

The above data elements (product code, serial number, batch number, and expiration date) are printed on a pack of the medicament in the form of a machine-readable two-dimensional code (e.g. data matrix code). The pack and the medicament contained therein form an individual pack that is individualized by the unique identifier.

The data elements of the unique identifier (at least the product code and the serial number) are additionally stored in a database (verification database). When an individual pack is dispensed to a patient, e.g. in a pharmacy, the pharmacist must first read the two-dimensional code with a reading device. This elicits a check of the product code and serial number against the verification database; the check examines whether the combination of product code and serial number exists and whether the individual pack with this combination has already been logged in the verification database as “dispensed”. If the combination of product code and serial number exists and the individual pack has not yet been “dispensed”, the pharmacist may dispense it to patients. If, after positive confirmation, the individual pack is dispensed, the status of the individual pack is set to “dispensed”. If a second individual pack having the identical combination of product code and serial number were now to be subjected to verification, it would be clear that this had already been dispensed. If an individual pack undergoing verification were found to have a combination of product code and serial number that does not exist in the verification database, this would mean it must be counterfeit.

Further requirements and information on the unique identifier are given in the Delegated Regulation and in the references cited therein, the content of which is incorporated into this description by reference.

The unique identifier makes it possible to identify a single individual pack. Since a single individual pack is normally used by just one person (particularly when it is a prescription-only medicament), use of the unique identifier likewise makes it possible to identify the owner of the individual pack to which the unique identifier is applied.

On the basis of the unique identifier it is thus possible to verify that a person is in possession of the corresponding medicament on the packaging of which the unique identifier is applied. This person is normally a patient who is taking the medicament. Accordingly, this person is a prospective participant in a drug trial in connection with the medicament.

Only the actual person in possession of the unique identifier is able to make use of the unique identifier. The unique identifier on the individual pack thus effectively serves as a personal admission ticket that can be used for enrolment of the person in one or more drug trials.

The unique identifier or a unique feature derived therefrom may additionally be used as a unique identifier of the person as a participant in one or more drug trials.

The unique identifier can here be used to determine from the product code the corresponding medicament and optionally the indication for which the medicament is licensed.

From the determined medicament and/or the determined indication, it is possible to check whether a drug trial having the determined medicament and/or the determined indication as its subject is being carried out or is planned.

In some embodiments, a prospective participant in a drug trial is in a first step provided (preferably by the sponsor of the drug trial) with a portal via which the prospective participant can be informed whether a drug trial is planned for which participants are being sought and/or in which he/she could take part.

The portal can be provided to a prospective participant for example in the form of a web portal on a computer. The computer may be the personal computer of the prospective participant. The computer may for example be designed as a desktop PC, portable PC, laptop, notebook, netbook, tablet PC or handheld (e.g. smartphone).

The web portal may be accessed with a web browser, for example via a specific internet address. It is also possible for the portal to be accessed in the form of a software application, the software application being installed on the computer and executable therefrom.

It is also possible for the portal to be accessed via a terminal (kiosk) situated in a hospital or pharmacy.

It is also possible for the tasks described in this description that are executed by a computer to be divided between more than one computer. It is for example possible for the prospective participant to have a computer that is connected to the server of the sponsor or of the investigator via one or more networks (for example, a cellular network, a WLAN, and/or the internet). Data inputted by the participant on his/her computer are transmitted via the portal to the server.

In a first step, a prospective participant in possession of an individual pack of a medicament having a unique identifier is prompted to input data of the unique identifier into the computer via the portal.

The data to be inputted comprise at least the product code and the serial number. The data may be inputted by the user in the form of alphanumeric characters, for example by means of an input device such as a keyboard, a mouse or a touchscreen. It is also possible for a microphone in combination with speech-recognition software to be used for inputting the data.

The prospective participant preferably inputs the data using a reading device, with which the machine-readable two-dimensional code of the unique identifier, which includes the product code and serial number, can be read in automatically. The reading device may be a component of the computer via which the portal is accessed or it may be connected to said computer (via a cabled or cable-free connection, e.g. via Bluetooth or WLAN). The reading device is configured to read a unique identifier. Since the unique identifier is according to Delegated Regulation (EU) 2016/161 printed on the packaging of the medicament in the form of a machine-readable two-dimensional code (e.g. as a QR code or data matrix code), the reading device may for example be designed as a camera. A camera is normally a component of smartphones and tablet computers. The reading device may also be a separate scanner/a separate camera for two-dimensional codes.

The two-dimensional code is interpreted by the reading device and/or the computer. The interpretation decodes the two-dimensional code of the unique identifier into alphanumeric characters.

In a further step, the product code and the serial number can be extracted from the alphanumeric characters.

In a further step, it is possible to verify that the prospective participant is in possession of the individual pack of the medicament. This check is able to verify whether the combination of product code and serial number exists in the verification database. If the combination of product code and serial number exists in the verification database, the individual pack is deemed to be the “original individual pack”. If the combination of product code and serial number does not exist in the verification database, the individual pack is deemed to be “counterfeit”. In addition, the status of the original individual pack can be determined. If the status is set as “dispensed”, the individual pack is deemed to be “legally obtained”. If the status is set as “not dispensed”, the individual pack is deemed to be “not legally obtained”.

In some embodiments, prospective participants receive information on one or more drug trials and/or are able to enroll and/or register in a drug trial only if they are in possession of an original individual pack.

In some embodiments, prospective participants receive information on one or more drug trials and/or are able to enroll and/or register in a drug trial only if they are in possession of a legally obtained individual pack.

In some embodiments, prospective participants receive information on one or more drug trials and/or are able to enroll and/or register in a drug trial only if they are in possession of a legally obtained and original individual pack.

In a further step, the medicament may be specified on the basis of the product code. In addition, it is possible to determine from the product code the pharmaceutical form, the strength, the pack size, and the type of packaging of the medicament.

The information may be read from a database (medicaments database) via the product code; said database may be a component of the computer or it may be connected to the computer via one or more networks. The medicaments database and the verification database may be a single database or different databases.

On the basis of the specified medicament and any further information on the medicament, a (further) database (drug trials database) can be searched for drug trials for which participants are being sought and/or in which a person taking the specified medicament could take part. The drug trials database may be a component of the computer or it may be connected to the computer via one or more networks. The drug trials database and the medicaments database may be a single database or different databases.

It is for example possible to read from the drug trials database all planned or ongoing drug trials of one or more sponsors in which the specified medicament is a study subject.

It is possible to read from the drug trials database all drug trials that are investigating indications in which the specified medicament is being used or could be used.

It is possible to use, for identification of the drug trial for which the prospective participant could be a suitable participant, further information on the prospective participant and his/her condition, for example age, sex, height, body weight, symptoms, diagnosed condition, pre-existing conditions, and/or the like. This patient information is preferably retrieved, and entered by the prospective participant, via the portal.

The identified drug trial(s) in which the prospective participant could take part is/are displayed to the prospective participant. Such information may be displayed on screen for example in the form of text optionally accentuated by graphic elements or images. The screen may be a component of the computer or be connected thereto. Voice output via a speaker is also possible.

It is preferable that the prospective participant is able to register for one or more drug trials via the portal. By registering, the prospective participant declares his/her consent to take part in the drug trial and to the processing of the data collected. Normally, he/she gives his/her consent by means of a so-called “informed consent”. He/she can give this informed consent via the portal, for example by means of a digital or electronic signature. It is also possible for a corresponding document to be displayed to him/her via the portal that he/she can print out with a printer, sign, and mail to the sponsor or investigator, or that he/she can scan into the computer with the aid of a document scanner and then transmit via the portal to the server.

In some embodiments, the participant is during registration for a drug trial linked to an individual identifier derived from a combination of product code and serial number of the unique identifier.

According to Delegated Regulation (EU) 2016/161, the unique identifier comprises a numeric or alphanumeric sequence of not more than 20 characters, generated by a deterministic or non-deterministic randomization algorithm (the serial number). From the combination of the product code and the serial number, it is possible to unambiguously identify an individual pack of a medicament. If this combination of product code and serial number (or an individual identifier derived from the combination) is linked to the (prospective) participant in possession of the individual pack, the (prospective) participant can be unambiguously identified from this combination (or from an individual identifier derived from the combination). This means it is not necessary to record and store personal data such as the name and address of the (prospective) participant, which is advantageous for data protection reasons. Instead, the results of the drug trial can be unambiguously assigned to a participant on the basis of the combination of product code and serial number (or of an individual identifier derived from the combination).

The combination of product code and serial number may be the sequence product code/serial number, the sequence serial number/product code, a nesting of product code and serial number, or some other linking of product code and serial number. It is also possible for the product code and serial number to be subjected to a (mathematical) algorithm that generates a unique identifier from the product code and serial number. It is for example possible to generate a hash value or the like. It is also possible for further data besides the product code and the serial number to be included in the generation of a unique identifier. It is in addition possible for not the entire product code and/or the entire serial number to be used for the generation of a unique identifier; it is merely necessary to ensure that the unique identifier distinguishes a participant in a drug trial from other participants.

In the drug trial, the respective participant can then be administered under the combination of product code and serial number or under the derived unique identifier.

The combination of product code and serial number, and the derived unique identifier, are preferably a numeric or alphanumeric code (character string).

However, it is also possible for the participant in one or more drug trials to be administered under a unique identifier that is generated when the portal is installed and/or is accessed for the first time and/or when registering for a (or the first) drug trial.

It is preferable that a participant in different drug trials is administered under the same unique identifier.

The use of a unique identifier in more than one drug trial has the advantage that the results from the more than one study can be related to one another and cross-study analyses carried out.

In a preferred embodiment, results of the drug trial are recorded via the portal. The English-language literature uses the term “patient reported outcome” (PRO) as a generic term for many different concepts for the measurement of subjectively perceived states of health. The common basis of said concepts is that the participant's state of health is assessed and reported by the participant him/herself. It is for example possible that, for the purposes of the drug trial, the participant fills out a health self-assessment questionnaire regularly and/or at defined times. It is preferable that questions or fields to be filled out are displayed to the participant via the portal and that he/she is able to answer or fill out said questions or fields with the aid of an input device (for example a keyboard, a mouse, a touchscreen, a microphone and/or the like). It is possible to use a chatbot to make it easier for the patient to input information.

It is possible for a participant to receive instructions during the drug trial via the portal. Such instruction can for example be a prompt to take a dose of the medicament, to perform a measurement (blood glucose measurement, blood pressure measurement, core body temperature measurement, body weight measurement, and/or the like), to answer one or more questions, to carry out a health self-assessment, and/or the like. Instructions may be displayed on the participant's computer in the form of text (optionally accentuated by graphic elements or images). Such instruction can for example be accompanied by a vibration alarm or an acoustic signal so that the participant is informed that an instruction has arrived.

It is possible for payment to a patient for taking part in the drug trial to be processed via the portal. Payment can for example be in the form of money or the reimbursement of travel expenses, food or grocery vouchers, or other services. It is possible via the portal for bank data for transfers to be sent, voucher codes (coupons) to be sent, cryptocurrencies to be paid out, invoices to be transmitted, times (e.g. for filling out questionnaires) to be recorded, and the like.

Some embodiments of the present disclosure include:

1. A process comprising the following steps:
inputting of data of a unique identifier affixed to an individual pack of a medicament into a computer by a prospective participant in a drug trial,
identifying, on the basis of the inputted data, one or more drug trials in which the prospective participant may take part,
displaying the one or more drug trials to the prospective participant.

2. The process according to embodiment 1, comprising the following steps:
extracting a product code and a serial number from the data of the unique identifier,
identifying, on the basis of the product code, a medicament and/or an indication,
identifying, on the basis of the identified medicament and/or the identified indication, one or more drug trials in which the prospective participant may take part,

3. The process according to embodiment 1 or 2, comprising the following steps:
extracting a product code and a serial number from the data of the unique identifier,
verifying, on the basis of the product code and the serial number, that the individual pack is an original and/or legally obtained individual pack.

4. The process according to embodiment 3, wherein one or more drug trials in which he/she may take part are displayed to the prospective participant only if the individual pack is an original and/or legally obtained individual pack.

5. The process according to any of embodiments 1 to 4, wherein the prospective participant captures a machine-readable two-dimensional code of the unique identifier using a reading device for machine-readable two-dimensional codes, the captured code is transmitted from the reading device to a computer, and the computer extracts a product code and a serial number from the captured code.

6. The process according to any embodiments 1 to 5, comprising the steps of:
inputting of patient information into the computer by the prospective participant,
identifying one or more drug trials with the aid of the patient information.

7. The process according to any embodiments 1 to 6, comprising the step of:
providing a portal for enrolling participants in a drug trial on the computer, the computer being operated by the prospective participant, the prospective participant inputting the data of the unique identifier and patient information into the computer via the portal, and information on one or more drug trials in which he/she may take part being displayed to the prospective participant via the portal.

8. The process according to embodiment 7, comprising the steps of:
registering the prospective participant as a participant in an identified drug trial,
capturing results of the drug trial for which the participant is registered, via the portal, in particular through inputting of information on the participant's state of health by the participant.

9. The process according to any of embodiments 1 to 8, comprising the steps of:
inputting of data of a unique identifier on an individual pack of a medicament into a computer by a prospective participant in a drug trial,
verifying, by checking the genuineness of the unique identifier on the basis of the inputted data, that the prospective participant is in possession of an original individual pack of the medicament,
identifying, on the basis of the inputted data, one or more drug trials in which the prospective participant may take part,
displaying the one or more identified drug trials to the prospective participant.

10. The process according to any of embodiments 1 to 9, comprising the steps of:
capturing on a first computer by a prospective participant in a drug trial, with the aid of a reading device, of a machine-readable, two-dimensional code of a unique identifier on an individual pack of a medicament,
determining a product code and a serial number from the two-dimensional code,
transmitting the product code and the serial number from the first computer to a second computer,
determining a medicament and/or an indication on the basis of the transmitted product code and with the aid of a medicaments database in which information on the medicament is stored under the product code,
identifying, with the aid of a drug trials database, one or more drug trials in which the determined medicament and/or the determined indication are a subject of the trial,
transmitting the one or more identified drug trials from the second computer to the first computer,
displaying the transmitted one or more identified drug trials on the first computer to the prospective participant.

11. A system comprising
an input unit,
an output unit,
a control unit, and
a database,
   wherein the control unit is configured to cause the input unit to receive data of a unique identifier affixed to an individual pack of a medicament from a prospective participant in a drug trial,
   wherein the control unit is configured to identify one or more drug trials in the database on the basis of the data received,
   wherein the control unit is configured to cause the output unit to display information on the one or more identified drug trials to the prospective participant.

12. The system according to embodiment 11, comprising
a first computer having a reading device and an output unit, and
a second computer connected to a medicaments database, to a drug trials database, and to a verification database,
   wherein the first computer is configured
to capture a machine-readable two-dimensional code of a unique identifier with the aid of the reading device,
to extract from the code a product code and a serial number,
to transmit the product code and the serial number to the second computer via a network,
   wherein the second computer is configured
to receive the product code and the serial number from the first computer via the network,
to verify that information on an individual pack of a medicament is stored in the verification database, wherein the product code and the serial number are assigned to the individual pack,
to identify a medicament and/or an indication in the drug trials database on the basis of the product code,
to identify one or more drug trials in the drug trials database on the basis of the identified medicament and/or the identified indication,
to send information on the one or more identified drug trials to the first computer via the network,
   wherein the first computer is configured
to receive information on the one or more identified drug trials from the second computer via the network,
to display the information on the one or more identified drug trials via the output unit.

13. The system according to embodiment 12,

wherein the first computer is configured to receive patient information and/or information on the health self-assessment of a participant in a drug trial via the input unit and to send it to the second computer via a network.

14. A computer program product comprising a data carrier and program code that is stored on the data carrier and that causes a computer, in the memory of which the program code has been loaded, to execute the following steps:
receiving data of a unique identifier applied to an individual pack of a medicament,
identifying one or more drug trials on the basis of the received data,
displaying information on the one or more drug trials to a prospective participant in the one or more drug trials.

15. The use of a unique identifier affixed to the individual pack of a medicament for the recruitment of participants for a drug trial, characterized in that a prospective participant inputs the data of the unique identifier into a computer, on the basis of the data one or more drug trials in which the prospective participant may take part are identified, and information on the one or more drug trials is displayed to the prospective participant.

### BRIEF DESCRIPTION OF THE DRAWINGS

FIG. 1 shows a schematic representation of an example of a unique identifier in accordance with Delegated Regulation (EU) 2016/16 of the European Commission, according to some embodiments;

FIG. 2 shows an individual pack of a medicament on which the unique identifier from FIG. 1 is printed, according to some embodiments;

FIG. 3 shows a schematic representation a system, according to some embodiments;

FIG. 4 shows a schematic representation in flowchart form of a process that may be executed with a system such as that shown in FIG. 3, according to some embodiments;

FIG. 5 shows a schematic representation of a system, according to some embodiments;

FIG. 6 shows a schematic representation in flowchart form of a the process that may be executed with a system such as that shown in FIG. 5, according to some embodiments;

FIG. 7 shows a schematic representation a system, according to some embodiments;

FIG. 8 shows a schematic representation in flowchart form of a process that may be executed with a system such as that shown in FIG. 7, according to some embodiments;

FIG. 9 shows a system of the disclosure such as that shown in FIG. 7 together with an individual pack of a medicament such as that shown in FIG. 2 having a unique identifier that bears a machine-readable two-dimensional code, according to some embodiments;

FIG. 10 shows a schematic representation in flowchart form of a process that may be executed with a system such as that shown in FIG. 3, 5, 7, or 9, according to some embodiments;

FIG. 11 shows a schematic representation in flowchart form of optional steps in a process that may be executed with a system such as that shown in FIG. 3, 5, 7, or 9, according to some embodiments;

FIG. 12 shows a schematic representation in flowchart form of optional steps in a process that may be executed with a system such as that shown in FIG. 3, 5, 7, or 9, according to some embodiments; and

FIG. 13 shows a schematic representation in flowchart form of optional steps in a process that may be executed with a system such as that shown in FIG. 3, 5, 7, or 9, according to some embodiments.

### DETAILED DESCRIPTION

The disclosure is elucidated in detail hereinbelow with reference to figures, without any intention to restrict the disclosure to the features or combinations of features shown in the figures.

FIG. 1 shows a schematic representation of an example of a unique identifier in accordance with Delegated Regulation (EU) 2016/16 of the European Commission.

The unique identifier (1) comprises alphanumeric characters (plain text) such as a product code (2), a serial number (3), a batch number (4), and an expiration date (5).

The product code specifies the medicament. For medicaments marketable in Germany, the so-called pharmacy product number (PPN) serves as the product code.

The unique identifier shown in FIG. 1 (1) also includes a data matrix code (6) in which the product code (2), the serial number (3), the batch number (4), and the expiration date (5) are encoded in machine-readable form.

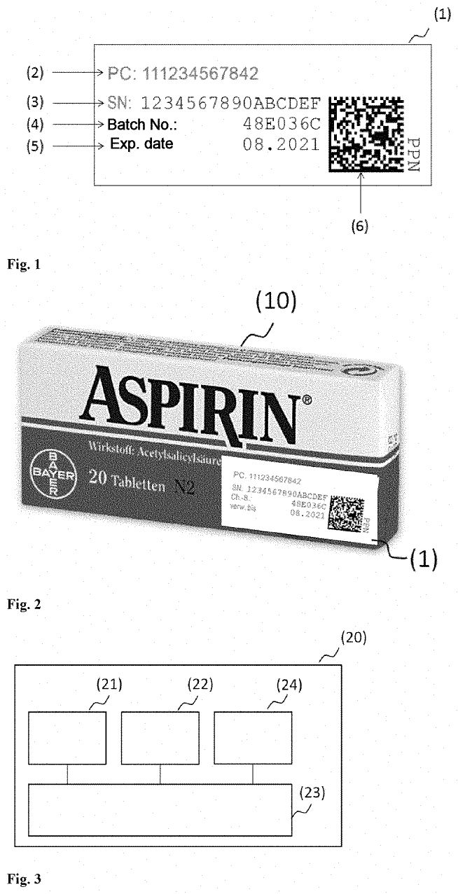

FIG. 2 shows an individual pack (10) of a medicament on which the unique identifier (1) from FIG. 1 is printed.

FIG. 3 shows a schematic representation of some embodiments of the system of the disclosure. The system (20) comprises an input unit (21), an output unit (22), a control unit (23), and a database (24).

The control unit (23) is configured to cause the input unit (21) to receive data of a unique identifier applied to the individual pack of a medicament from a prospective participant in a drug trial and, on the basis of the received data, to identify one or more drug trials in the database (24) and to cause the output unit (22) to display information on the one or more identified drug trials to the prospective participant.

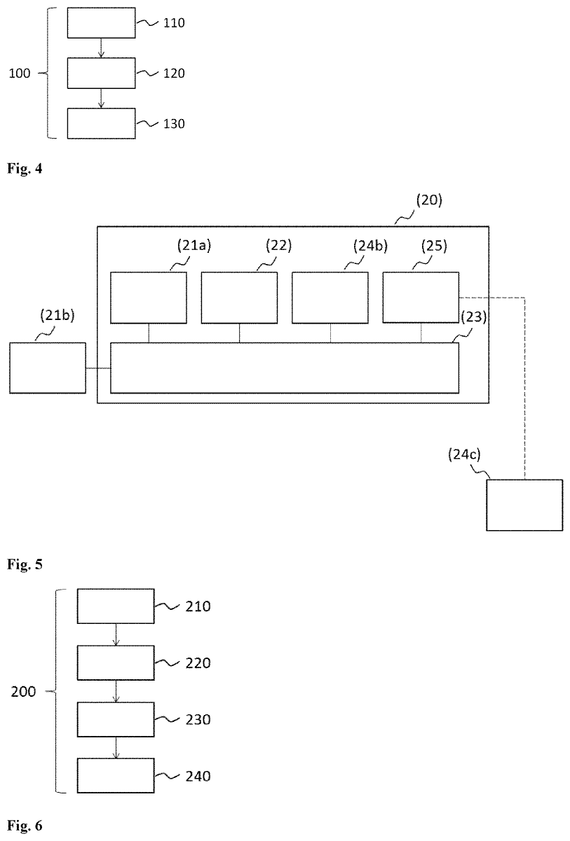

FIG. 4 shows a schematic representation in flowchart form of some embodiments of the process of the disclosure that may be executed with the system shown in FIG. 3.

The process (100) comprises the steps of:
(110) inputting of data of a unique identifier affixed to an individual pack of a medicament into a computer by a prospective participant in a drug trial,
(120) identifying, on the basis of the inputted data, one or more drug trials for which participants are being sought and/or in which the prospective participant may take part,
(130) displaying the one or more drug trials to the prospective participant.

FIG. 5 shows a schematic representation of some embodiments of the system of the disclosure. The system (20) comprises an input unit (21a), a reading device (21b) for machine-readable two-dimensional codes, an output unit (22), a control unit (23), a drug trials database (24b), and a transmitter and receiver unit (25).

The reading device (21b) is normally used by a prospective participant in a drug trial to capture the machine-readable two-dimensional code of a unique identifier on an individual pack of a medicament. The control unit (23) is configured to receive the captured data from the reading device (21b). Depending on the design, the captured data may be a recorded digital image of the two-dimensional code or it may be the alphanumeric data (product code, serial number, and the like) decoded therefrom. When this is a recorded digital image of the two-dimensional code, the control unit (23) is configured to decode the two-dimensional code.

Control commands and/or further data may be inputted via the input unit (21a).

The control unit (23) is configured to extract the product code and the serial number from the data received by the reading device (21b). The control unit (23) is configured to verify that the prospective participant is in possession of the individual pack of the medicament. In this check, the control unit (23) verifies that the combination of product code and serial number exists in the verification database (24c). The control unit (23) may in addition be configured to verify that the original individual pack had been legally obtained. In this check, the control unit (23) verifies that the status “dispensed” is entered for the individual pack in the verification database (24c).

The control unit (23) is additionally configured to identify one or more drug trials in the drugs trials database (24b) on the basis of the data received from the reading device (in particular on the basis of the product code), and to cause the output unit (22) to display information on the one or more identified drug trials to the prospective participant.

FIG. 6 shows a schematic representation in flowchart form of some embodiments of the process of the disclosure that may be executed with the system shown in FIG. 5.

The process (200) comprises the steps of:
(210) inputting of data of a unique identifier affixed to an individual pack of a medicament into a computer by a prospective participant in a drug trial with the aid of a reading device for machine-readable two-dimensional codes,
(220) verifying that the prospective participant is in possession of an original individual pack of the medicament, by checking the genuineness of the unique identifier on the basis of the inputted data,
(230) identifying, on the basis of the inputted data, one or more drug trials for which participants are being sought and/or in which the prospective participant may take part,
(240) displaying the one or more identified drug trials to the prospective participant.

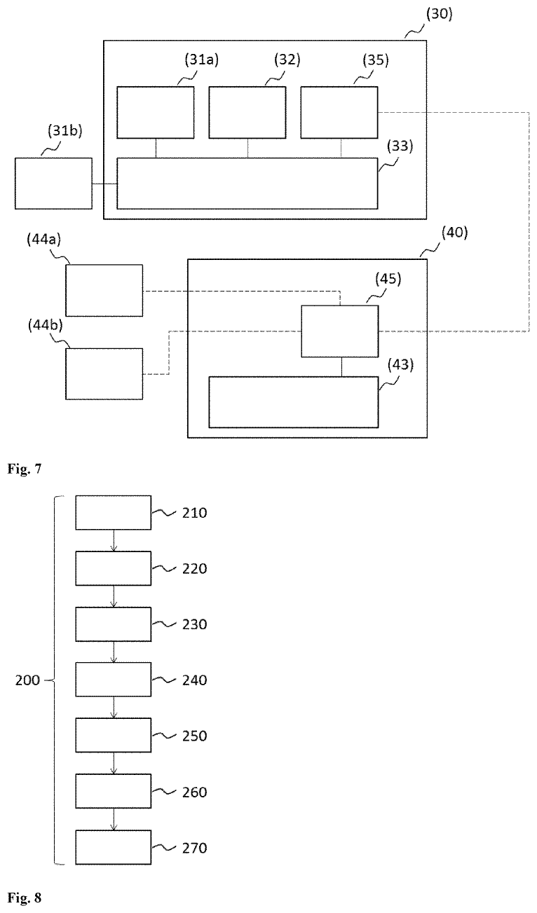

FIG. 7 shows a schematic representation of some embodiments of the system of the disclosure. The system (20) comprises a first computer (30), a second computer (40), a reading device (31b) for two-dimensional, machine-readable codes, a medicaments database (44a), and a drugs trials database (44b).

The first computer (30) comprises an input unit (31a), an output unit (32), a control unit (33), and a transmitter and receiver unit (35).

The second computer (40) comprises a control unit (43) and a transmitter and receiver unit (45).

The first computer (30) and the second computer (40) are connected to one another via one or more networks (represented by the dashed line between the transmitter and receiver unit (35) of the first computer (30) and the transmitter and receiver unit (45) of the second computer (40)).

The second computer (40) is connected to the medicaments database (44a) and the drug trials database (44b) via one or more networks (represented by the dashed line between the transmitter and receiver unit (45) of the second computer (40) and the medicaments database (44a) and the drug trials database (44b)).

The reading device (31b) is connected to the first computer (30) and is normally used by a prospective participant in a drug trial to capture the machine-readable two-dimensional code of a unique identifier on an individual pack of a medicament and to send the captured data to the first computer (30). The control unit (33) of the first computer (30) is configured to receive the captured data from the reading device (31b). Depending on the design, the captured data may be a recorded digital image of the two-dimensional code or it may be the alphanumeric data (product code, serial number, and the like) decoded therefrom. When this is a recorded digital image of the two-dimensional code, the control unit (33) of the first computer (30) is configured to decode the two-dimensional code.

Control commands and/or further data (e.g. patient data and/or information on the health self-assessment) may be inputted into the computer (30) via the input unit (31a) of the first computer (30).

Information may be outputted to the prospective participant via the output unit (32) of the first computer (30).

Data may be sent from the first computer (30) to the second computer (40), and data may be received by the second computer (40), via the transmitter and receiver unit (35) of the first computer (30).

Data may be received by the first computer (30), and data may be sent from the second computer (40) to the first computer (30), via the transmitter and receiver unit (45) of the second computer (40). In addition, data from the medicaments database (44a) and from the drug trials database (44b) may be read via the transmitter and receiver unit (45) of the second computer (40).

It is possible for the medicaments database (44a) to be a combined database in which a medicaments database and a verification database are combined in one database. It is possible for the control unit (43) of the second computer (40) to be configured to verify that the individual pack is a legally obtained, original individual pack.

FIG. 8 shows a schematic representation in flowchart form of some embodiments of the process of the disclosure that may be executed with the system shown in FIG. 7.

The process (300) comprises the steps of:
(310) capturing on a first computer by a prospective participant in a drug trial, with the aid of a reading device, of a machine-readable, two-dimensional code of a unique identifier on an individual pack of a medicament,
(320) determining a product code and a serial number from the two-dimensional code,
(330) transmitting the product code and the serial number from the first computer to a second computer,
(340) determining a medicament and/or an indication on the basis of the transmitted product code and with the aid of a medicaments database in which information on the medicament is stored under the product code,
(350) identifying, with the aid of a drug trials database, one or more drug trials in which the determined medicament and/or the determined indication are a subject of the trial,
(360) transmitting the one or more identified drug trials from the second computer to the first computer,
(370) displaying the transmitted one or more identified drug trials on the first computer to the prospective participant.

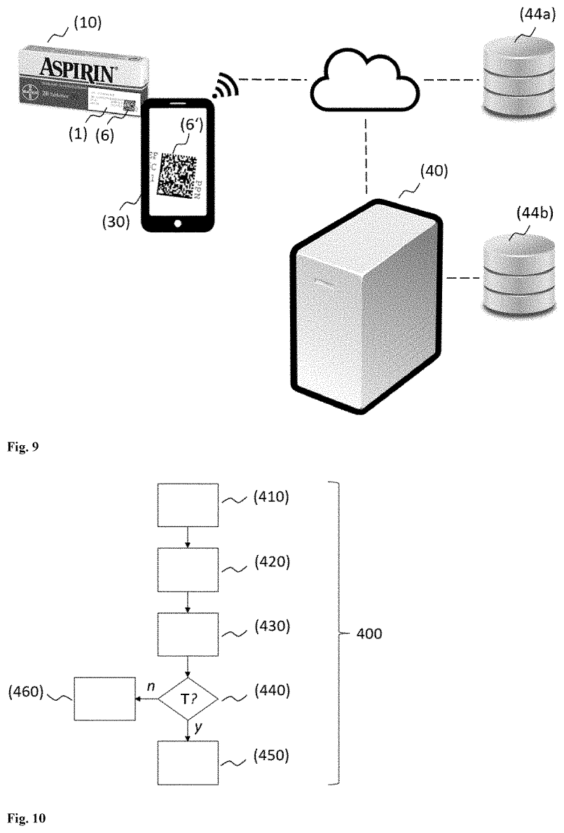

FIG. 9 shows, by way of example, some embodiments of the system of the disclosure shown in FIG. 7 together with the individual pack (10) of a medicament shown in FIG. 2 having a unique identifier (1) that bears a machine-readable two-dimensional code (6).

The first computer (30) is designed as a smartphone. The camera normally present in a smartphone is used as a reading device for the machine-readable two-dimensional code. The camera generates an image (6′) of the machine-readable two-dimensional code, which is displayed on a screen of the smartphone. The smartphone is configured so as to be able to decode the two-dimensional code. This results in identification of the product code and the serial number. These data may be sent to the second computer (40) via a cellular network and the internet (depicted by the cloud). The second computer (40) is designed as a server. The server is connected to the medicaments database (44a) and to the drug trials database (44b) via various networks. From the product code, the server in the medicaments database (44a) is able to determine information about the medicament and the indication in which it is being used. From the determined medicament or the determined indication, the server in the drug trials database (44b) is able to identify one or more drug trials having the indication and/or the medicament as their subject. Information on the drug trial may be transmitted from the server, via the internet and the cellular network, to the smartphone, where it is displayed on screen to the prospective participant.

It is possible for the medicaments database (44a) to be a combined database in which a medicaments database and a verification database are combined in one database. It is possible for the control unit (43) of the second computer (40) to be configured to verify that the individual pack is a legally obtained, original individual pack.

FIG. 10 shows a schematic representation in flowchart form of some embodiments of the process of the disclosure that may be executed with the systems shown in FIGS. 3, 5, 7, and 9.

The process (400) comprises the steps of:
(410) capturing, by a prospective participant in a drug trial using a camera, of a unique identifier applied to an individual pack of a medicament in the form of a two-dimensional optically readable code,
(420) extracting a product code and a serial number from the captured unique identifier.
(430) determining a medicament from a medicaments database on the basis of the product code,
(440) checking whether a planned or ongoing drug trial (T) is stored in a drug trials database for the product code and/or the determined medicament,
(450) if a planned or ongoing drug trial (T) is stored in the drug trials database for the product code and/or the determined medicament: displaying information on the drug trial to the prospective participant,
(460) if no planned or ongoing drug trial (T) is stored in the drug trials database for the product code and the determined medicament: displaying a message to the prospective participant that there is currently no planned or ongoing drug trial for the medicament.

FIG. 11 shows a schematic representation in flowchart form of further optional steps in the process of the disclosure that may be executed with the systems shown in FIGS. 3, 5, 7, and 9.

The steps are:
(510) checking whether an entry (E) exists in a verification database for the product code and the serial number,
(520) if no entry (E) for the product code and the serial number exists in the verification database: displaying a message to the prospective participant that registration for a drug trial is not possible,
(530) if an entry (E) for the product code and the serial number exists in the verification database: checking whether the entry includes a notice (N) that the medicament has been marked as dispensed,
(540) if the medicament has not been marked as dispensed: displaying a message to the prospective participant that registration for a drug trial is not possible,
(550) if the medicament has been marked as dispensed: displaying a message to the prospective participant that registration for a drug trial is possible.

Steps (510) to (550) may, by way of example, take place after step (330) or (350) or (420) or (450).

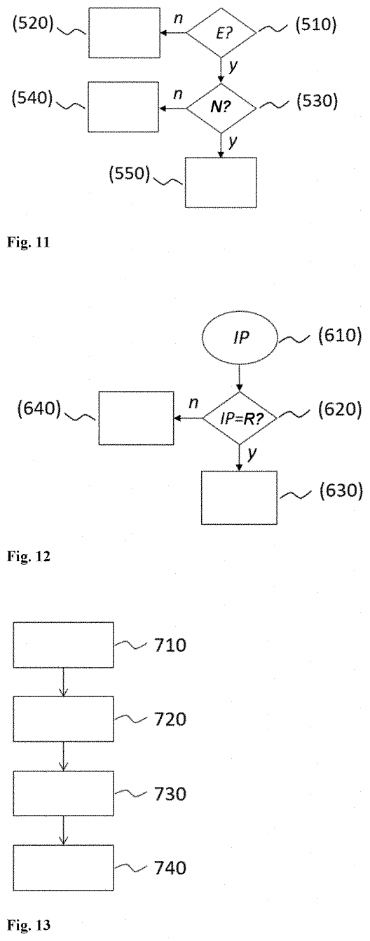

FIG. 12 shows a schematic representation in flowchart form of further optional steps in the process of the disclosure that may be executed with the systems shown in FIGS. 3, 5, 7, and 9.

The steps are:
(610) receiving information (IP) about the prospective participant from the prospective participant, the information including age, sex, height, body weight, symptoms, diagnosed condition, and/or pre-existing conditions,
(620) comparing the information on the prospective participant with the requirements (R) for a drug trial stored in the drug trials database,
(630) if the information (IP) on the prospective participant meets the requirements (R): displaying information on the drug trial to the prospective participant and/or prompting the prospective participant to register for the drug trial,
(640) if the information (IP) on the prospective participant does not meet the requirements (R): displaying a message to the prospective participant that there is currently no planned or ongoing drug trial for which he/she can register.

Steps (610) to (640) may, by way of example, take place after step (240) or (370) or (450) or (550).

FIG. 13 shows a schematic representation in flowchart form of further optional steps in the process of the disclosure that may be executed with the systems shown in FIGS. 3, 5, 7, and 9.

The steps are:
(710) generating an individual identifier for the prospective participant using the product code and/or the serial number,
(720) recording the prospective participant as a participant in the drug trial in a database under the individual identifier,
(730) receiving data on the health of the participant in the drug trial during the drug trial,
(740) storing the health data in the database under the individual identifier.

Steps (710) to (740) may, by way of example, take place after step (370) or (450) or (550) or (630).

1. A process comprising:
inputting data of a unique identifier affixed to an individual pack of a medicament into a computer by a prospective participant in a drug trial;
identifying one or more drug trials on the basis of the inputted data; and
displaying the one or more drug trials to the prospective participant.

2. The process of claim 1, comprising:
extracting a product code and a serial number from the data of the unique identifier;
identifying, on the basis of the product code, a one or more of a medicament or an indication; and
identifying, on the basis of the one or more of the identified medicament or the identified indication, one or more drug trials.

3. The process claim 1, comprising:
extracting a product code and a serial number from the data of the unique identifier; and
verifying, on the basis of the product code and the serial number, that the individual pack is an original and/or legally obtained individual pack.

4. The process of claim 3, wherein one or more drug trials are displayed to the prospective participant only if the individual pack is one or more of an original or a legally obtained individual pack.

5. The process claim 1, wherein the prospective participant captures a machine-readable two-dimensional code of the unique identifier using a reading device for machine-readable two-dimensional codes, the captured code is transmitted from the reading device to a computer, and the computer extracts a product code and a serial number from the captured code.

6. The process of claim 1, comprising:
inputting of patient information into the computer by the prospective participant; and
identifying one or more drug trials with the aid of the patient information.

7. The process of claim 1, comprising:
providing a portal for enrolling participants in a drug trial on the computer, the computer being operated by the prospective participant, the prospective participant inputting the data of the unique identifier and patient information into the computer via the portal, and information on one or more drug trials being displayed to the prospective participant via the portal.

8. The process of claim 7, comprising:
registering the prospective participant as a participant in an identified drug trial; and
capturing results of the drug trial for which the participant is registered, via the portal, in particular through inputting of information on the participant's state of health by the participant.

9. The process of claim 1, comprising:
inputting of data of a unique identifier on an individual pack of a medicament into a computer by a prospective participant in a drug trial;
verifying, by checking the genuineness of the unique identifier on the basis of the inputted data, that the prospective participant is in possession of an original individual pack of the medicament;
identifying one or more drug trials on the basis of the inputted data; and
displaying the one or more identified drug trials to the prospective participant.

10. The process of claim 1, comprising:
capturing on a first computer by a prospective participant in a drug trial, with the aid of a reading device, of a machine-readable, two-dimensional code of a unique identifier on an individual pack of a medicament;
determining a product code and a serial number from the two-dimensional code;
transmitting the product code and the serial number from the first computer to a second computer;
determining a medicament and/or an indication on the basis of the transmitted product code and with the aid of a medicaments database in which information on the medicament is stored under the product code;
identifying, with the aid of a drug trials database, one or more drug trials in which the determined medicament and/or the determined indication are a subject of the trial;
transmitting the one or more identified drug trials from the second computer to the first computer; and
displaying the transmitted one or more identified drug trials on the first computer to the prospective participant.

11. A system comprising:
an input unit;
an output unit;
a control unit; and
a database,
wherein the control unit is configured to cause the input unit to receive data of a unique identifier affixed to an individual pack of a medicament from a prospective participant in a drug trial,
wherein the control unit is configured to identify one or more drug trials in the database on the basis of the data received,
wherein the control unit is configured to cause the output unit to display information on the one or more identified drug trials to the prospective participant.

12. The system of claim 11, comprising:
a first computer having a reading device and an output unit; and
a second computer connected to a medicaments database, to a drug trials database, and to a verification database,
wherein the first computer is configured
to capture a machine-readable two-dimensional code of a unique identifier with the aid of the reading device;
to extract from the code a product code and a serial number; and
to transmit the product code and the serial number to the second computer via a network,
wherein the second computer is configured
to receive the product code and the serial number from the first computer via the network;
to verify that information on an individual pack of a medicament is stored in the verification database, wherein the product code and the serial number are assigned to the individual pack;
to identify a medicament and/or an indication in the drug trials database on the basis of the product code;
to identify one or more drug trials in the drug trials database on the basis of the identified medicament and/or the identified indication; and
to send information on the one or more identified drug trials to the first computer via the network,
wherein the first computer is configured
to receive information on the one or more identified drug trials from the second computer via the network; and
to display the information on the one or more identified drug trials via the output unit.

13. The system of claim 12,
wherein the first computer is configured to receive one or more of patient information or information on the health self-assessment of a participant in a drug trial via the input unit and to send it to the second computer via a network.

14. A computer program product comprising a data carrier and program code that is stored on the data carrier and that causes a computer, in the memory of which the program code has been loaded, to execute the following steps:
receiving data of a unique identifier applied to an individual pack of a medicament;
identifying one or more drug trials on the basis of the received data; and
displaying information on the one or more drug trials to a prospective participant in the one or more drug trials.

15. (canceled)
